(19) United States
(12) Patent Application Publication
(10) Pub. No.: US 2007/0015968 A1
(43) Pub. Date: Jan. 18, 2007

(54) Apparatus and method for fluid propulsion of an elongate device

(76) Inventor: Judson Shelnutt, Athens, GA (US)

Correspondence Address: SHUMAKER LOOP & KENDRICK, 101 E. KENNEDY, SUITE 2800, TAMPA, FL, 33672-0609, US

(21) Appl. No.: 11/485,098

(22) Filed: Jul. 12, 2006

Related U.S. Application Data
(60) Provisional application No. 60/698,578, filed on Jul. 13, 2005.

(51) Int. Cl. A61B 1/12

(52) U.S. Cl. 600/156; 600/129; 600/108

(57) ABSTRACT

An elongate, fluid propelled tube functional as an endoscope for performing numerous medical procedures. The elongate tube is sufficiently thin to penetrate difficult-to-reach areas. The elongate tube can be controlled by a remote control that drives one or more fluid pumps.

### RELATED APPLICATIONS

This disclosure claims priority to U.S. Provisional Application Ser. No. 60/698,578, entitled “APPARATUS AND METHOD FOR FLUID PROPULSION OF AN ELONGATE DEVICE,” which was filed on Jul. 12, 2005.

### TECHNICAL FIELD

This disclosure relates to medical devices, and more particularly, to elongate devices such as endoscopes, catheters, and the like.

### BACKGROUND INFORMATION

Regular endoscopic examinations of internal structures such as the esophagus, lungs, colon, uterus, and other organ systems are known to provide major public health benefits. Conventional endoscopes typically include an imaging system and a light source. The imaging system transmits images from the tissue adjacent to the distal end of the endoscope to a monitor or display. The fiber optic light illuminates the tissue in the path of the endoscope. In addition, most endoscopes include one or more working channels through which medical devices such as biopsy forceps, snares, fulguration probes, and other tools may be passed. Control cables similar to puppet strings are carried within the endoscope body and connect a flexible portion of the distal end to a set of control knobs at the proximal endoscope handle. By manipulating the control knobs, the physician steers the distal end of the endoscope during insertion and directs it to the region of interest.

In use, for example in an esophageal procedure, a physician inserts the endoscope into a patient by positioning the distal end of the endoscope adjacent the mouth. The physician then delivers the distal end of the endoscope into the patient's esophagus. In particular, to insert the endoscope, the physician grips the proximal end of the endoscope and manually drives it distally. As a result, the physician relies in large part on the axial rigidity or columnar strength of the endoscope in order to push the endoscope into the patient's esophagus. To facilitate insertion, the physician can steer the distal end of the endoscope as it is inserted into the patient.

Despite many years of modifications and improvements, traditional endoscopes have a number of drawbacks. One significant drawback is the size of typical endoscopes. While the outside diameter of endoscopes has been reduced over time, endoscopes continue to have large outside diameters. Endoscopes having such large outside diameters simply cannot be used to access small diameter lumens. Moreover, the large diameter of such endoscopes greatly increases the discomfort experienced by patients during endoscopic procedures.

Another drawback is the lack of flexibility of conventional endoscopes. This results from the relatively inflexible and sturdy components used in endoscopes, including the control cables that are utilized for steering. Moreover, the axial rigidity necessary to push the endoscope distally into a body lumen has prevented manufacturers of endoscopes from moving to truly flexible and small-diameter endoscopes. Moreover, due to the size and limited flexibility of conventional endoscopes, they are often clumsy, non-intuitive, and cause a substantial amount of friction during use. These drawbacks greatly increase patient discomfort. Another common complaint about traditional endoscopes is the limited operator control of stiffness along the scope length.

Yet another drawback is that conventional endoscopes are expensive medical devices costing in the range of $25,000 for an endoscope, and much more for the associated operator console. Because of the expense, these endoscopes are built to withstand repeated disinfections and use upon many patients. Conventional endoscopes are generally built of sturdy materials, which decreases the flexibility of the scope and thus can decrease patient comfort. Furthermore, conventional endoscopes are complex and fragile instruments which can frequently need expensive repair as a result of damage during use or during a disinfection procedure.

Similar problems plague other elongate medical devices such as catheters, feeding tubes, cannulas, and the like. To overcome these and other problems, there is a need for a highly flexible endoscope that has better navigation and tracking, improved access by reduced factional forces upon the lumenal tissue, increased patient comfort, and greater clinical productivity and patient throughput than those that are currently available.

### BRIEF SUMMARY

Accordingly, one purpose of the present invention is to provide an endoscope, endoscope system, and method of accessing a small diameter body cavity. The endoscope, system, and method have features that resolve or improve upon one or more of the above-described drawbacks of conventional endoscopes.

In a first aspect of the present invention, the endoscope includes a tubular body having a proximal portion, a distal end, and a central axis extending therebetween. A central passageway extends between the proximal portion and the distal end. A fiber optic shaft is disposed within the central passageway. The fiber optic shaft is operably connected to an imaging system, which allows a physician to visualize the tissues in the vicinity of the endoscope distal end. In addition to the central passageway, the tubular body of the endoscope includes at least two propulsion passageways. The propulsion passageways are offset from the central axis of the tubular body. The propulsion passageways end at a distal port that is configured to expel fluids laterally and proximally relative to the central axis, thereby propelling the endoscope distally. Fluid pumps are operably connected to the propulsion passageways. A control interface allows a physician to steer and propel the endoscope toward a desired direction. The endoscope has a total outside diameter between about 4 millimeter and 10 millimeters.

In a second aspect of the present invention, the endoscope includes a laser operably connected to the distal end of the tubular body.

In a third aspect of the present invention, the endoscope includes an electrocautery conduit disposed along the exterior of the tubular body. An energy source is then connected to the electrical conduit. As such, the electrocautery conduit is energizable to cauterize tissue.

In a fourth aspect of the present invention, a method includes accessing a small diameter body lumen. The small diameter body lumen can include a portion of a patient's gastrointestinal tract, ureter(s), intracranial space, and/or vascular system. The method utilizes a small diameter endoscope having a distal propulsion system and an imaging system. The method includes the steps of passing the distal end of the tubular body into the body lumen. Once inside the body lumen, one or more fluid pumps are used to propel the distal end of the tubular body relative to the body lumen. The physician can then visualize the body lumen with the imaging system.

In a fifth aspect of the present invention, a method includes accessing the fallopian tube of a patient. The method utilizes a small diameter endoscope having a distal propulsion system and an imaging system. The method involves accessing the patient's cervical ostium with an introducer sheath. The distal end of the endoscope can then be passed through the introducer sheath and into the cervical ostium. Once inside the cervical ostium, the propulsion system can be used by the physician to propel the distal end of the tubular body relative to the cervical ostium. The physician steers the distal end of the endoscope into the fallopian tube and can then image the fallopian tube with the imaging system. A laser disposed within a central passageway of the endoscope can also be used within the fallopian tube, for example to eliminate or reduce the size of a blockage, or to ligate the fallopian tube. Alternatively, an sclerosing agent can be injected through the central lumen of the endoscope to ligate the fallopian tube.

In a sixth aspect of the present invention, a method includes accessing the peritoneum of a patient via a fallopian tube by utilizing a small diameter endoscope having an imaging system. The method involves accessing the patient's cervical ostium with an introducer sheath. The distal end of the endoscope can then be passed through the introducer sheath and into the cervical ostium. Once inside the cervical ostium, the physician steers the distal end of the endoscope into the fallopian tube and can then image the fallopian tube with the imaging system. When the distal end of the endoscope is inserted into the fallopian tube, the endoscope can be inserted the entire length of the fallopian tube and into the peritoneal cavity. Diagnostics or other medical procedures can then be performed within the peritoneal cavity.

### BRIEF DESCRIPTION OF THE DRAWINGS

Embodiments of the present invention will now be described by way of example with reference to the accompanying drawings, in which:

FIG. 1 illustrates a side view of an endoscope system according to one aspect of the present invention;

FIG. 2 illustrates a cross sectional side view of the distal portion of an endoscope according to one aspect of the present invention;

FIG. 3 illustrates a cross sectional view of the tubular body of an endoscope according to one aspect of the present invention;

FIG. 4 illustrates a side view of the tubular body of an endoscope according to one aspect of the present invention;

FIG. 5 illustrates a plan view of a user control interface according to one aspect of the present invention;

FIG. 6 illustrates a side view of a user control interface according to one aspect of the present invention;

FIG. 7 illustrates a method of treating tubal factor and infertility; and

FIG. 8 illustrates a method of performing a tubal ligation.

While the embodiments of the invention will be described in connection with the preferred embodiment, it will be understood that it is not intended to limit the invention to that embodiment. On the contrary, it is intended to cover all alternatives, modifications, and equivalents, as may be included within the spirit and scope of the invention as defined by the claims.

### DETAILED DESCRIPTION OF THE PRESENTLY PREFERRED EMBODIMENTS

The invention is described with reference to the drawings in which like elements are referred to by like numerals. The relationship and functioning of the various elements of this invention are better understood by the following detailed description. However, the embodiments of this invention as described below are by way of example only, and the invention is not limited to the embodiments illustrated in the drawings. It should also be understood that the drawings are not to scale and in certain instances, details which are not necessary for an understanding of the present invention, such as conventional details of fabrication and assembly, have been omitted.

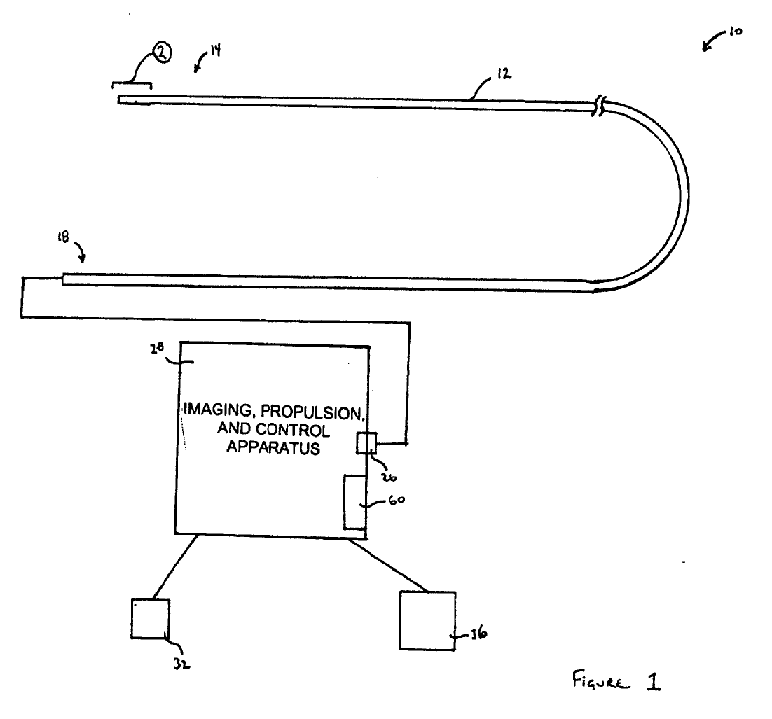

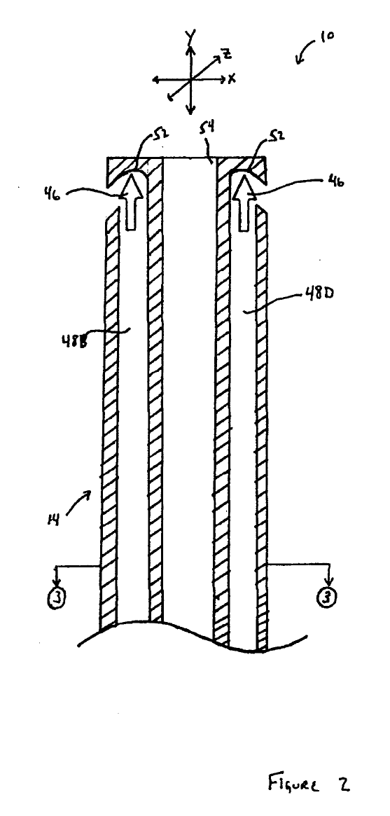

Referring to the drawings, FIG. 1 illustrates a first embodiment of the present invention, and in particular, a remote-controlled, flexible, small-diameter endoscope system 10. More particularly, endoscope system 10 includes a tubular body 12 having a distal end 14 and a proximal end 18. A plurality of propulsion passageways 48 extend along tubular body 12 and exit the endoscope at directional nozzles or ports, which are located at distal end 14. A central passageway 54 is provided to house imaging and illumination fiber optics, as well as a laser accessory. The proximal end 18 of the endoscope is connected to an imaging, propulsion, and control apparatus 28. Pumps 60 are provided in the imaging, propulsion, and control apparatus 28 to propel fluid through propulsion passageways 48 (FIG. 2) in order to drive and steer the endoscope. In addition, imaging, propulsion, and control apparatus 28 is connected to a monitor 36 and a user control interface 32 having a joystick for pump operation. During use, monitor 36 displays images from the body lumen adjacent the endoscope distal end. The body lumen is illuminated by an illumination fiber optic bundle 44. A variety of accessories, including laser and cautery, can also be added to the endoscope system. In use, a physician can navigate the endoscope through small-diameter body lumens by using the joystick and concurrently viewing the tissues adjacent the distal end of the endoscope on the monitor.

As illustrated in FIG. 1, endoscopic system 10 includes a flexible, small-diameter tubular body 12 having a distal portion 14, and a proximal portion 18. The tubular body includes propulsion passageways and a central passageway, which we discussed in detail below. Tubular body 12 can be formed from a unitary structure having a plurality of passageways for example by a conventional extruding process. It can alternatively be formed from a plurality of shafts enclosed by an outer sheath. The tubular body 12 can be formed from a flexible material such as soft plastic, e.g., silastic tubing, or any other suitable highly flexible polymer material. Preferably, tubular body 12 has an outside diameter between about 1 mm and 10 mm. The relatively small outside diameter allows the tubular body to be inserted into a wide variety of relatively small body lumens.

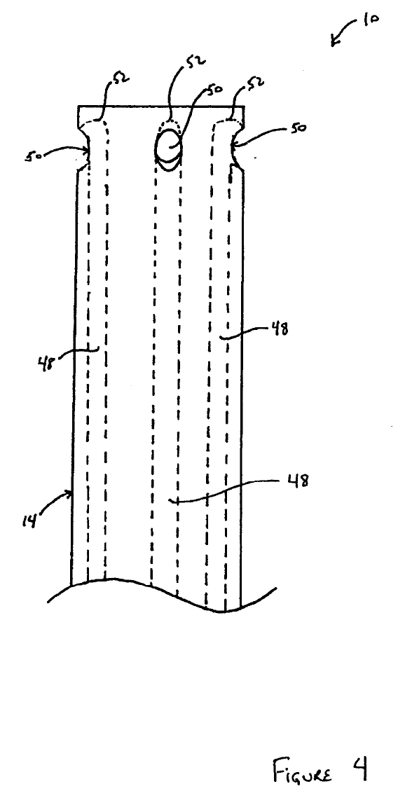

As illustrated in FIGS. 2 and 4, endoscopic system 10 includes propulsion passageways 48, which extend along the length of tubular body 12. While the embodiments illustrated at FIGS. 2 and 4 include four propulsion passageways, additional or fewer passageways can alternatively be provided. At the distal end of the endoscope, the propulsion passageways terminate in directional nozzles or propulsion ports 50. The ports 50 direct the propulsion fluid in a lateral and proximal direction in order to drive and steer the endoscope as described below. The tubular body also includes a central passageway 54 that houses the optics, illumination fibers, and accessories.

The proximal end of the tubular body 12 is connected to the imaging, propulsion, and central apparatus 28. In particular, the propulsion passageways 48 are connected via connector 26 (FIG. 1) to one or more pumps 60 in the imaging, propulsion, and control apparatus 28. The pumps are sufficiently powerful to drive the endoscope as described below. Exemplary irrigation pumps include the ECO PMP™, available through Olympus or the NEZHAT-DORSEY™ irrigation pump. The pumps are controlled by user control interface 32, as described in greater detail below. The pumps, in turn, are connected to a fluid reservoir or saline solution source. In particular, pump 60 can be directly attached to a fluid source such as a water reservoir located in imaging, propulsion, and control apparatus 28. Alternatively, a water or saline solution hook-up can be provided on imaging propulsion and control apparatus 28 so that the endoscopic system 10 can be hooked-up to a faucet.

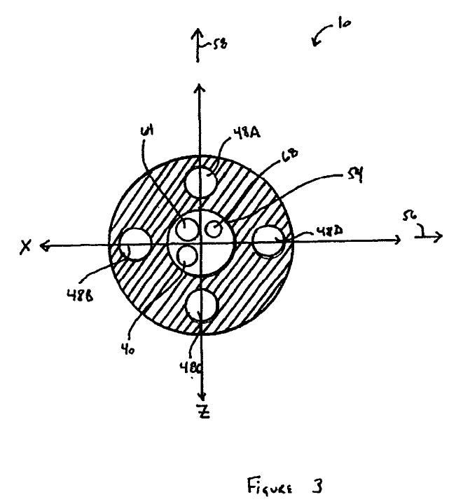

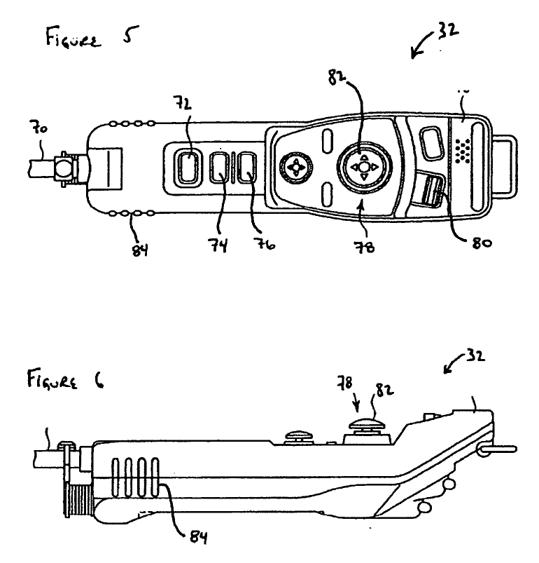

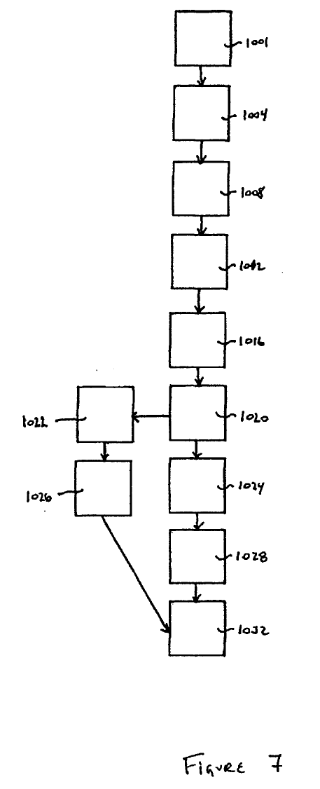

When the pumps are activated, fluid is propelled into propulsion passageways 48 and exits through nozzles or propulsion ports 50, thus driving the endoscope. In particular, as the fluid is propelled through propulsion passageways 46, the fluid creates a force 46 against distal surface 52. This force causes the distal end of endoscopic system 10 to move distally relative to axis Y. Moreover, changing the velocity of fluid passing through passageways 48 laterally deflects distal end 14 with respect to axis Y. For example, when the velocity of the fluid passing through 48B (FIG. 3) exceeds the velocity of the fluid passing through 48D, the distal end of endoscope 10 is deflected in direction 56. Conversely, when the velocity of the fluid passing through passageway 48D exceeds the velocity of the fluid passing through passageway 48B, the distal end of endoscope 10 is deflected in a direction 84. Likewise, when the velocity of the fluid passing through passageway 48 A exceeds the velocity of the fluid passing through passageway 48C, the distal end of endoscope 10 is deflected in the direction opposite to direction 58. Conversely, when the velocity of the fluid passing through passageway 48C exceeds the velocity of the fluid passing through passageway 48A, the distal end of endoscope 10 is deflected in direction 58. As such, by altering the velocity of the fluid propelled through various passageways 48, a user can selectively guide endoscope 10 laterally or distally. Such navigation is accomplished by using joystick 82, which is shown at FIGS. 5-6. In addition, to move the endoscope 10 proximally the physician can manually retract or withdraw the endoscope 10.

Alternatively, an elongate tube having a single propulsion passageway can be provided, as illustrated in FIG. 9. The single propulsion passageway can be used to “self-center” the elongate tube as it moves distally along a body lumen. Once at a target location, the passageway can be used to deliver a desired fluid, nutrient, or pharmaceutical agent to the patient. As described below, the device can thus be used as a feeding tube, for example. In the feeding tube embodiment, the diameter can be relatively large compared to other embodiments described herein.

In one embodiment, a pump 60 can be provided for each of the passageways 48A-D. Alternatively, a single pump 60 can provide propulsion for all of the propulsion passageways 48A-D. In this case, valves can be used to direct the fluid into the desired passageways. In either case, the operation of these pumps is controlled by user control interface 32, as described in greater detail below.

As illustrated in FIG. 2, endoscopic system 10 is provided with a central passageway 54. Central passageway 54 is configured to house at least one luminous conductor 62 for light conduction and illumination. In particular, the luminous conductor 62 extends from the connector 26 of the imaging, propulsion, and control apparatus 28 to the distal end 14 through the central passageway 54. The luminous conductor 62 can be formed from a fiber optic light carrying strand or bundle. The connector 26 provides a connector, as understood by those skilled in the art, between the luminous conductor 62 and a conventional light source, which is housed in the imaging, propulsion, and control apparatus 28. Light travels through the connector 26 and along the tubular body 12 via the illumination fiber optic bundle 44. It should be noted that one or more imaging fiber optic bundles 40 may be provided in order to increase the amount of light or intensity of light provided by the illumination fiber optic bundle 44. An adjustable light valve (not shown) for selectively adjusting the intensity of the light, may also be provided.

As illustrated in FIGS. 2-3, central passageway 54 is also configured to house an optical conductor 64. Optical conductor 64 gathers and transmits the interior cavity image to the imaging, propulsion, and control apparatus 28 and, ultimately, to the monitor 36. Referring to FIG. 3, an optical conductor 64 is shown. Optical conductor 64 is interfaced with the imaging, propulsion, and control apparatus 28 via connector 26. The imaging, propulsion, and control apparatus 28, in turn, relays the images gathered at the distal end of the endoscope to monitor 36. An imaging processor (not shown) can also be provided in order to capture the image gathered by the optical conductor 64. The gathered image can then be transferred to the monitor 36 and/or to video capable glasses (not shown). In one embodiment, an optical wedge (not shown) is included. The optical wedge can be located near the distal end 14 of endoscope 10 to provide a direction of view compensation of about 5° to 10° when viewed under water, as is necessary if implemented in a variety of medical procedures.

As illustrated in FIGS. 2 through 3, central passageway 54 can also be provided with a variety of accessories for use in various medical procedures. For example a laser 68 can be provided within central passageway 54. Laser 68 is connected to the imaging, propulsion, and control apparatus 28 by connector 26. A physician can activate laser 68 by using the laser control 72 on control interface 32, which is shown at FIGS. 5-6. In addition, a touhy-bourst connector or other injection port can be provided to inject medications through central passageway 54 to a target region. A cautery source may also be provided along exterior portion of distal end 14 of endoscope 10. An electrical conductor (not shown) is used to connect the cautery portion along distal end 14 to the imaging, propulsion, and control apparatus 28 via connector 26. The cautery portion can be selectively energized by user control interface 32, and in particular, cautery control 74, as illustrated in FIGS. 5-6.

As illustrated in FIGS. 5 through 6, the user control interface 32 is used to navigate, steer, and operate the endoscope system 10. User control interface 32 is provided with a switch 80 used to turn the endoscope system on or off. That the user control interface also includes a joystick 82 used to operate the fluid pumps and, in turn, the directional movements of the distal end 14 of the endoscopic system 10 as discussed above. Buttons 72, 74, and 76 are used to activate the laser, the cautery device, and the image capture trigger, respectively. An alternative user control interface 32 can be provided with wireless capabilities, including blue tooth, Wi-Fi, RF, or other wireless systems that are apparent to those of ordinary skill in the art. The user control interface 32 can also be configured as a traditional joystick. Moreover, the user control interface 32 can also be provided with additional or fewer control buttons depending on the number of accessories provided on the endoscope. In addition, as illustrated in FIGS. 5-6, handgrips 84 can also be provided along user control interface 32.

Referring to FIG. 1, a monitor 36 is provided for viewing images captured by the imaging fiber optic bundle. Monitor 36 may include one or more image display devices such a CRT, HDTV, plasma, or LCD type video display units. Although visual clarity is an important feature of the monitor 36, the invention is not limited to the quality of the examples provided above.

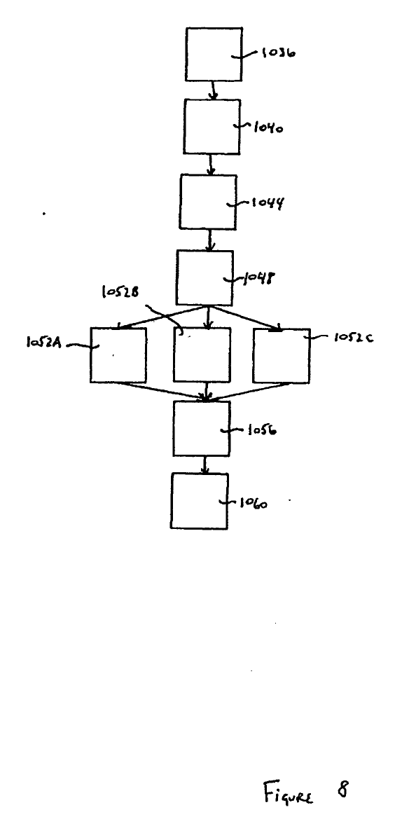

An embodiment of the present invention includes a method of performing medical procedures in very narrow, small diameter body cavities while under direct visual control via monitor 36. As shown in FIG. 7, one method of the present invention can be used to treat tubal factor infertility, which is presently untreatable. One embodiment of this method may include the step 1001 of positioning and preparing the patient in accordance with procedures well-known to those of skill in the art. Once the patient is positioned and prepared for the procedure, in step 1004 a speculum is used to open the vagina. At this point an introducer sheath (e.g., an empty sleeve) is used to access the cervical ostium, illustrated as step 1008. With the introducer sheath in place, in step 1012, the distal end of endoscope 10 can be passed via the introducer sheath into the uterus. When the distal end of the endoscope is in position in the uterus, in step 1016, the physician or user can operate the user control interface 32 to navigate the endoscope within the uterus. By observing the monitor 36, the physician can visualize the progress and position of the endoscope within the uterus. In step 1020, the physician steers the endoscope 10 towards the fallopian tube opening and into the fallopian tube of interest. Once inside the fallopian tubes the endoscope can be steered towards the fallopian tube blockage. In step 1024, the physician clears the blockage. To clear the blockage, a physician may activate the laser, or inflate a dilation balloon positioned at the distal end of the endoscope 10. If necessary, the physician can also deliver a self-expanding stent to prop open the previously blocked portion. Once the obstruction or blockage is opened or removed, the physician may proceed to the next blockage, if any, or retract the endoscope from the patient, as shown in step 1032.

It should be noted that the fallopian tubes, as well as the uterus, are potential spaces. These potential spaces are advantageously distended by the propulsion fluid delivered through the propulsion passageways in the endoscope. This facilitates visualization of the surrounding tissues as well as penetration of the fallopian tubes.

FIG. 8 illustrates use of the above-described endoscope to perform a tubal ligation. In a tubal ligation, step 1036 includes positioning and preparing the patient as described above. Likewise, in step 1040 a speculum is used to open the vagina, and in step 1044 an introducer sheath is used to access the cervical ostium. Once the endoscope is in the uterus, in step 1048 the physician may operate the user control interface to steer the endoscope distal end to the first fallopian tube. Once the endoscope distal end is about 6-8 centimeters into the fallopian tube, in step 1052A the physician may activate the laser by using laser button 72 (FIGS. 5-6) in order to ablate and obstruct the fallopian tube by laser cautery. Alternatively, in step 1052B a sclerosing agent can be used to obstruct the fallopian tube. In particular, the sclerosing agent, such as silver nitrate or another suitable acidic solution, can be injected into the fallopian tube via the central passageway. In another alternative, shown as step 1052C, a coil can be delivered to the fallopian tube site in order to obstruct the fallopian tube. In any case, once the first fallopian tube is obstructed, in step 1056 the physician can retract the distal end of the endoscope into the uterus and again operate joystick of the user control interface to navigate the distal end of the endoscope into the second fallopian tube. Once the endoscope has been navigated to an appropriate site with the fallopian tube, the physician may obstruct the second fallopian tube as described above in steps 1052A-C with respect to the first fallopian tube. Throughout this procedure, the physician may monitor the progress and position of the endoscope 10 by viewing monitor 36. Once both fallopian tubes are obstructed, in step 1060 the physician may retract the endoscope from the patient. It is also contemplated that other types of small diameter endoscopes could be used to access the fallopian tubes and similarly obstruct the fallopian tubes to treat tubal factor infertility, as described above.

Another method includes performing a diagnostic laparoscopy without the need to make an incision. In particular, the physician can enter the uterine cavity and the fallopian tubes as described above in relation to FIGS. 7 and 8. Since the fallopian tubes lead directly into the peritoneal cavity, in step 1022 (FIG. 7) a physician can navigate the endoscope into the peritoneal cavity and perform diagnostic procedures by visualizing the surrounding tissues in the peritoneal cavity on the monitor 36. Once the endoscope is inside the peritoneal cavity, in step 1026 the physician may use the endoscope to monitor the exterior of the uterus, the fallopian tubes, the ovaries, the peritoneal lining, the small bowel, the appendix, and even upper abdominal structures such as the liver or gall bladder. Thus, the endoscope 10 can be used to diagnose such ailments as endometriosis, pelvic inflammatory disease, ovarian cancer or appendicitis.

Other alternative methods include using the endoscope to perform a cytology wash, the delivery of radiation pellets (brachitherapy), the delivery of chemotherapeutic drugs, ovarian drilling, or egg harvesting.

The above described endoscope can further be used in a wide variety of non-obstetrical or gynecological procedures. For example one method includes using the endoscopic system 10 in a minimally invasive neurosurgery context. For example, the endoscopic system 10 can be used to treat occlusive hydrocephalus. In particular, to treat occlusive hydrocephalus the endoscopic system 10 is used to perform a ventriculostomy. Other operations include the treatment of intraventricular processes, e.g. arachnoidal cysts and cystic cerebral rumors, intraventricular hemorrhages and other intraventricular interventions. Other intracranial procedures include endoscopically assisted neurosurgery, i.e., via the base of the skull. Transnasal transsphenoidal pituitary gland surgery can likewise be performed using the endoscopic system 10. The endoscopic system 10 can further be used for traditional diagnostic endoscopy procedures, including colonoscopy, sigmoidoscopy, upper gastrointestinal endoscopy and for evaluation of the proximal small bowel. Likewise, endoscope system 10 can be utilized in urological procedures, such as the evaluation of large portions of the genitourinary tract, to treat stones, stenosis, or cancer in the upper tract.

In addition, larger diameter tubes can also be provided with the above-disclosed propulsion system. For example, a nasojejunal feeding tube having the above-disclosed propulsion system can be provided. The feeding tube can be navigated into the stomach of a patient. Once in the stomach, the feeding tube can be secured to the patient so as to limit further displacement of the feeding tube within the patient. When secured to the patient, the propulsion passageways can be used to deliver fluids, nutrients, or pharmaceutical agents to the patient.

The drawings and specification disclose a typical preferred embodiment of the invention, and although specific terms are employed, the terms are used in a descriptive sense only and not for purposes of limitation. The invention has been described in considerable detail with specific reference to these illustrated embodiments. It will be apparent, however, that various modifications and changes can be made within the spirit and scope of the invention as described in the foregoing specification. It is understood that other materials and dimensions may be used for the endoscopic type instrument of the present invention, keeping in mind the dimensions of the affected body parts. Further, the number and dimensions of the channels or passageways employed are variable depending on the accessories (i.e. laser, fiber optics, etc.) used in conjunction with the instrument. Further, the elongate medical tube described herein is not limited to endoscopes, or even medical devices. A wide variety of embodiments of the present invention will become apparent to those of skill in the art in view of the present disclosure. Accordingly, the invention is to be limited only by the scope of the appended claims.

What is claimed is:

1. An elongate medical device, comprising:
a tubular body having a proximal portion, a distal end, and a central axis extending therebetween;
a central passageway extending between the proximal portion and the distal end;
a fiber optic shaft disposed within the central passageway, the fiber optic shaft being operably connected to an imaging system;
a propulsion passageway extending between the proximal portion and a position proximal to the distal end, the propulsion passageway being offset from the central axis wherein the propulsion passageway comprises a distal port configured to expel fluids laterally relative to the central axis; and
a housing comprising a fluid pump operably connected to the propulsion passageway.

2. The elongate medical device of claim 1 wherein the distal port is further configured to expel fluids proximally relative to the central axis.

3. The elongate medical device of claim 2 wherein the central passageway and the propulsion passageway have a combined outside diameter between about 4 millimeters and 10 millimeters.

4. The elongate medical device claim 2 wherein the tubular body is formed from silastic tubing.

5. The elongate medical device of claim 1 further comprising a laser operably connected to the distal end of the tubular body.

6. The elongate medical device of claim 1 further comprising an electrocautery conduit disposed along the exterior of the tubular body, and an energy source operably connected to the electrical conduits, whereby the electrocautery conduit is energizable to cauterize tissue.

7. The elongate medical device of claim 1 further comprising an energy source and a light source disposed within the central passageway, the light source being operably connected to the energy source.

8. The elongate medical device of claim 1 further comprising second and third propulsion passageways, the second and third passageways having a distal port configured to expel fluids laterally relative to the central axis.

9. A method of accessing the fallopian tube of a patient, the method comprising the steps of;
a) providing an elongate medical device, comprising:
a tubular body having a proximal portion, a distal end, and a central axis extending therebetween;
a central passageway extending between the proximal portion and the distal end,
a fiber optic shaft disposed within the central passageway, the fiber optic shaft being operably connected to an imaging system;
first and second propulsion passageways extending between the proximal portion and a position proximal to the distal end, the propulsion passageways being offset from the central axis, wherein each of the propulsion passageways comprises a distal port configured to expel fluids laterally relative to the central axis; and
a housing comprising a fluid pump operably connected to the first propulsion passageway and a fluid pump operably connected to the second propulsion passageway;
b) providing an introducer sheath configured to receive the tubular body;
c) accessing the patient's cervical ostium with the introducer sheath;
d) passing the distal end of the tubular body through the introducer sheath and into the cervical ostium;
e) operating the fluid pump to propel the distal end of the tubular body relative to the cervical ostium;
f) steering the distal end of the tubular body into the fallopian tube by alternating activation of the fluid pumps; and
g) imaging the fallopian tube with the imaging system.

10. The method of claim 9, further comprising:
h) providing a laser within the central passageway;
i) locating a blockage within the fallopian tube; and
j) activating the laser so as to reduce the size of the blockage.

11. The method of claim 9, further comprising:
h) providing a port for delivering a fluid via the central passageway;
i) locating a target anatomy by using the imaging system; and
j) injecting a schelerosing agent through one of the central passageway, the first propulsion passageway, and the second propulsion passageway.

12. The method of claim 9, further comprising the steps of:
h) providing a port for delivering a fluid via the central passageway; and
i) injecting a chemotherapeutic agent through one of the central passageway, the first propulsion passageway, and the second propulsion passageway.

13. The method of claim 9, further comprising the steps of:
h) providing a port for delivering a fluid via the central passageway; and
i) locating a target anatomy; and
j) performing a cytology wash adjacent

14. The method of claim 9, further comprising the steps of:
h) providing a laser within the central passageway; and
i) activating the laser so as to cauterize the fallopian tube, thereby ligating the fallopian tube,

15. A method of accessing the peritoneum of a female patient, the method comprising the steps of:
a) providing an elongate medical device, comprising:
a tubular body having a proximal portion, a distal end, and a central axis extending therebetween;
a central passageway extending between the proximal portion and the distal end,
a fiber optic shaft disposed within the central passageway, the fiber optic shaft being operably connected to an imaging system;
first and second propulsion passageways extending between the proximal portion and a position proximal to the distal end, the propulsion passageways being offset from the central axis, wherein each of the propulsion passageways comprises a distal port configured to expel fluids laterally relative to the central axis; and
a housing comprising a fluid pump operably connected to the first propulsion passageway and a fluid pump operably connected to the second propulsion passageway;
b) providing an introducer sheath configured to receive the tubular body;
c) accessing the patient's cervical ostium with the introducer sheath;
d) passing the distal end of the tubular body through the introducer sheath and into the cervical ostium;
e) operating the fluid pump to propel the distal end of the tubular body relative to the cervical ostium;
f) steering the distal end of the tubular body into the fallopian tube by alternating activation of the fluid pumps;
g) imaging the fallopian tube with the imaging system;
h) maneuvering the distal end of the tubular body into the peritoneal cavity via the fallopian tube.

16. The method of claim 15 further comprising the step of:
i) imaging the peritoneal cavity.

17. The method of claim 16, further comprising the step of:
j) maneuvering the distal end of the tubular body to the appendix of the patient; and imaging the appendix.

18. The method of claim 15, further comprising the step of:
i) maneuvering the distal end of the tubular body to the ovary of the patient; and imaging the ovary.

19. A method of performing a medical procedure in a body lumen of a patient, the method comprising the steps of:
a) providing an elongate medical device, comprising:
a tubular body having a proximal portion, a distal end, and a central axis extending therebetween;
a central passageway extending between the proximal portion and the distal end,
a fiber optic shaft disposed within the central passageway, the fiber optic shaft being operably connected to an imaging system;
first and second propulsion passageways extending between the proximal portion and a position proximal to the distal end, the propulsion passageways being offset from the central axis, wherein each of the propulsion passageways comprises a distal port configured to expel fluids laterally relative to the central axis; and
a housing comprising a fluid pump operably connected to the first propulsion passageway and a fluid pump operably connected to the second propulsion passageway;
b) passing the distal end of the tubular body into the body lumen;
c) operating the fluid pump to propel the distal end of the tubular body relative to the body lumen;
f) steering the distal end of the tubular body by alternating activation of the fluid pumps; and
g) imaging the body lumen with the imaging system.

20. The method of claim 19 wherein the body lumen comprises a portion of the gastrointestinal tract.

21. The method of claim 19 wherein the body lumen comprises a portion of the ureter of the patient.

22. The method of claim 19 wherein the body lumen comprises a portion of the intracranial space of the patient.

23. The method of claim 19 wherein the body lumen comprises a portion of the vascular system of the patient.

24. A method of accessing the peritoneum of a female patient, the method comprising the steps of:
a) providing an endoscope having a proximal portion, a distal portion, and a portion therebetween, the endoscope having an outside diameter between about 4 millimeters and 10 millimeters, the endoscope having a control system adapted to deflect the distal end of the endoscope;
b) providing an introducer sheath configured to receive a distal portion of the endoscope;
c) accessing the patient's cervical ostium with the introducer sheath;
d) passing the distal end of the tubular body through the introducer sheath and into the cervical ostium;
e) operating the control system to deflect the distal end of the endoscope toward a fallopian tube of the patient;
g) maneuvering the distal end of the tubular body into the peritoneal cavity via the fallopian tube.

25. An elongated medical device comprising:
a shaft having a distal end and a proximal end;
a propulsion surface near the distal end of the shaft; and
a fluid passageway extending through the shaft, the fluid passageway configured to direct a fluid passing through the fluid passageway against the propulsion surface so as to generate a distally directed propulsion force in the distal end of the shaft sufficient to propel the elongate medical device in a distal direction.

26. The elongate medical device of claim 25, wherein the fluid passageway comprises an exit port through a side wall of the shaft adjacent to the propulsion surface for allowing the fluid to exit the shaft after contacting the propulsion surface.

27. The elongate medical device of claim 26, wherein the exit port is configured to generate a lateral force in the distal end of the shaft sufficient to propel the distal end of the elongate medical device in a lateral direction.

28. The elongate medical device of claim 25, wherein the shaft comprises a second passageway in fluid communication with the fluid passageway for allowing the fluid to pass proximally through the shaft after contacting the propulsion surface.

29. The elongate medical device of claim 25, wherein the fluid passageway comprises a plurality of fluid passageways circumferentially disposed about a cross-sectional area of the shaft.

30. The elongate medical device of claim 29, wherein the plurality of fluid passageways each comprise an exit port through a side wall of the shaft adjacent to the propulsion surface for allowing the fluid to exit the shaft after contacting the propulsion surface.

31. The elongate medical device of claim 30, wherein the exit ports are each configured to generate a lateral force in the distal end of the shaft sufficient to propel the distal end of the elongate medical device in a lateral direction.
